(19) United States
(12) Patent Application Publication
(10) Pub. No.: US 2022/0370165 A1
(43) Pub. Date: Nov. 24, 2022

(54) CONSOLE FOR CONTROLLING A ROBOTIC MANIPULATOR

(71) Applicant: CMR SURGICAL LIMITED, Cambridge, Cambridgeshire (GB)

(72) Inventors: Rebecca Anne Cuthbertson, Cambridge, Cambridgeshire (GB); Luke David Ronald Hares, Cambridge, Cambridgeshire (GB); Keith Marshall, Cambridge, Cambridgeshire (GB)

(21) Appl. No.: 17/771,088

(22) PCT Filed: Oct. 21, 2020
(86) PCT No.: PCT/GB2020/052637

§ 371 Date: Apr. 22, 2022

(30) Foreign Application Priority Data
Oct. 22, 2019 (GB) 1915269.3

(51) Int. Cl. A61B 34/00; A61B 34/37; A61B 34/20; B25J 13/06; G05G 9/047

(52) U.S. Cl. CPC A61B 34/74; A61B 34/37; A61B 34/20; B25J 13/065; G05G 9/047; A61B 2034/742; A61B 2090/035

(57) ABSTRACT

A console for controlling a robotic manipulator having an end effector, the console comprising: a hand controller connected to a gimbal assembly; and an articulated linkage connected at its proximal end to a rigid support structure, and at its distal end to the gimbal assembly. The gimbal assembly comprises only three degrees of freedom provided by only three joints, a first joint of the three joints permitting the gimbal assembly to rotate relative to the distal end of the articulated linkage about a first axis. The articulated linkage and gimbal assembly are arranged such that in every configuration of the articulated linkage and gimbal assembly, the first axis has the same orientation relative to the support structure. The articulated linkage has a parallelogram profile thereby mechanically constraining the first axis to have the same orientation relative to the support structure in every configuration of the articulated linkage.

### FIELD OF THE INVENTION

This invention relates to consoles for controlling robotic systems such as master-slave manipulators.

### BACKGROUND

Master-slave manipulators typically comprise a slave device for performing an action, and a master device which is directly manipulated by a user. The master device and slave device are operatively coupled such that the user's manipulation of the master device causes the slave device to perform a corresponding action. Master-slave manipulators are common in many technical fields, for example in the field of surgical robotics, in which a surgeon at a console manipulates hand controllers to cause a surgical robot to perform an operation.

FIG. 1 illustrates a known controller for a master-slave manipulator having an end effector that comprises a pair of moveable jaws. The controller has a primary input stem 101. The primary input stem constitutes the distal end of a gimbal assembly 102. The proximal end of the gimbal assembly is attached to a support structure of a console by a linkage, part of which is shown at 103. The primary input stem is provided with two rotatable elements 104, 105 which can be bound by loops 106 to a user's fingers. The user can move the primary input stem 101 to command a change in position of the end effector, and can move the elements 104, 105 to command opening or closing of the jaws of the end effector. The gimbal assembly 102 has four degrees of rotational freedom. This enables the gimbal assembly to accommodate motion of the primary input stem in three degrees of rotational freedom with a kinematic redundancy. Use of the redundant joint enables the gimbal assembly to avoid the kinematic singularity that would otherwise result when motion of the primary input stem causes two of the rotational axes of the gimbal assembly to become aligned. This controller is relatively large, which can be problematic when the workspace of the controller is limited.

This problem is exacerbated when the user is manipulating two such controllers in a common workspace, one in each hand.

### SUMMARY OF THE INVENTION

According to a first aspect, there is provided a console for controlling a robotic manipulator having an end effector, the console comprising: a hand controller connected to a gimbal assembly; and an articulated linkage connected at its proximal end to a rigid support structure, and at its distal end to the gimbal assembly; wherein the gimbal assembly comprises only three degrees of freedom provided by only three joints, a first joint of the three joints permitting the gimbal assembly to rotate relative to the distal end of the articulated linkage about a first axis; and wherein the articulated linkage and gimbal assembly are arranged such that in every configuration of the articulated linkage and gimbal assembly, the first axis has the same orientation relative to the support structure.

The console may be configured such that when the console is located on a horizontal surface, the first axis is vertical in every configuration of the articulated linkage and gimbal assembly.

The console may be configured to wholly accommodate rotation of the hand controller by articulation of the three joints of the gimbal assembly.

The console may be configured to accommodate translation of the hand controller by articulation of the articulated linkage.

The gimbal assembly may comprise: a first link and a second link; a second joint permitting the first link to rotate relative to the second link about a second axis, the second axis being perpendicular to the first axis; and a third joint permitting the hand controller to rotate relative to the second link about a third axis, the third axis being perpendicular to the second axis.

From a central position of the gimbal assembly in which the first axis, second axis and third axis are all perpendicular to each other, the range of motion of the first joint may be limited such that it is capable of rotating more than 90° in either rotational direction about the first axis.

From the central position of the gimbal assembly, the first joint may be limited to a maximum rotation angle of between 90° and 115° in a rotational direction which causes the first link to move towards the distal end of the articulated linkage.

From the central position of the gimbal assembly, the first joint may be limited to a maximum rotation angle of between 90° and 100° in a rotational direction which causes the first link to move away from the distal end of the articulated linkage.

From a central position of the gimbal assembly in which the first axis, second axis and third axis are all perpendicular to each other, the range of motion of the second joint may be limited such that it is capable of rotating less than 90° in either rotational direction about the second axis.

From the central position of the gimbal assembly, the second joint may be limited to a maximum rotation angle of between 80° and 90° in a rotational direction which causes the second link to move towards the first link.

From the central position of the gimbal assembly, the second joint may be limited to a maximum rotation angle of between 80° and 90° in a rotational direction which causes the second link to move away from the first link.

From a central position of the gimbal assembly in which the first axis, second axis and third axis are all perpendicular to each other, the range of motion of the third joint may be limited such that it is capable of rotating less than or the same as 90° in either rotational direction about the third axis.

From the central position of the gimbal assembly, the third joint may be limited to a maximum rotation angle of 90° in either rotational direction about the third axis.

The articulated linkage may have a parallelogram profile thereby mechanically constraining the first axis to have the same orientation relative to the support structure in every configuration of the articulated linkage.

The console may further comprise a position sensor located at the first joint for measuring a yaw motion of the hand controller solely by sensing a rotation of the first joint about the first axis.

The console may further comprise a position sensor located at the second joint for measuring a pitch motion of the hand controller solely by sensing a rotation of the second joint about the second axis.

The console may further comprise a position sensor located at the third joint for measuring a roll motion of the hand controller solely by sensing a rotation of the third joint about the third axis.

The console may be a surgeon's console for controlling a surgical robot carrying a surgical instrument.

The console may further control a further robotic manipulator having a further end effector. The console may further comprise: a further hand controller connected to a further gimbal assembly; and a further articulated linkage connected at its proximal end to the rigid support structure, and at its distal end to the further gimbal assembly; wherein the further gimbal assembly comprises only three degrees of freedom provided by only three joints, a first joint of the three joints permitting the further gimbal assembly to rotate relative to the distal end of the further articulated linkage about a fourth axis; and wherein the further articulated linkage and the further gimbal assembly are arranged such that in every configuration of the further articulated linkage and the further gimbal assembly, the fourth axis has the same orientation relative to the support structure.

The hand controller may be configured for operation by one hand of a user, and the further hand controller may be configured for operation by the other hand of the user.

### BRIEF DESCRIPTION OF THE FIGURES

The present invention will now be described by way of example with reference to the accompanying drawings. In the drawings:

FIG. 1 illustrates a known controller for a master-slave manipulator;

FIG. 2 illustrates a master-slave manipulator;

FIG. 3 illustrates an input device of a console for controlling a robotic manipulator; and

FIG. 4 illustrates a hand controller and gimbal assembly of a console.

### DETAILED DESCRIPTION

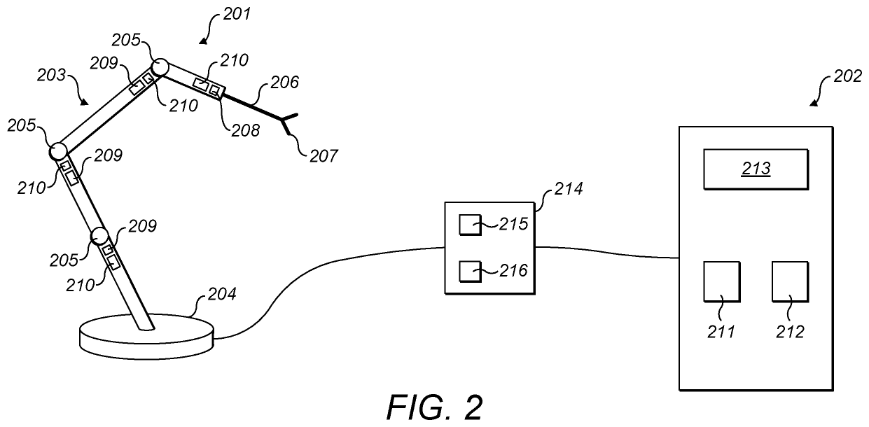

FIG. 2 illustrates schematically the general architecture of a master-slave manipulator in which a robot shown generally at 201 is controlled by a console shown generally at 202. The robot 202 comprises a robot arm 203 which extends from base 204. The robot arm is articulated by a series of revolute joints 205 along its length. The distal end of the robot arm 203 is connected to an instrument 206. The instrument 206 terminates in an end effector 207. In this example the end effector has a pair of opposed jaws. These can be moved relative to each other to grip or cut objects located between the jaws. The end effector is driven to move by a motor 208 at the distal end of the robot arm. The motor 208 is coupled to the end effector by cables extending along the interior of the instrument's shaft. The joints of the robot arm are driven to move by motors 209. These motors may be distributed along the arm. Each motor may be located proximal to the joint it is driving. Position sensors and force/torque sensors 210 may be located on the robot arm to sense the position of the joints and the forces/torques acting on the joints 205.

The console 202 comprises an input device 211 which is manipulated by a user to cause manipulation of the robot arm 203 and instrument 206. The console may also comprise a second input device 212. One input device may be configured for operation by one hand of a user for manipulating one robot arm, and the other input device may be configured for operation by the other hand of the user for manipulating another robot arm. The console may further comprise a display screen 213 for enabling the user to view the manipulation being performed by the instrument 206.

Control unit 214 controls the robot arm 203 in response to control inputs. The control unit 214 receives control inputs from input device 211. The control unit 214 may also receive control inputs from other sources, such as position sensor and force/torque sensors 210. The control unit 214 comprises a processor 215 which executes code stored in a non-transient form in a memory 216. On executing the code, the processor 215 determines a set of signals for commanding movement of the joints of the robot, and for moving the end effector 207 of the instrument in dependence on the inputs from the input device 211 and the robot arm position/force sensors 210. Control unit 214 may be located at the console 202, at the robot arm 203 or elsewhere in the system.

The master-slave manipulator system illustrated in FIG. 2 may, for example, be a surgical robotic system. In this example, console 202 is a surgeon's console, and the robot 201 is a surgical robot carrying a surgical instrument 206 for performing surgery. The surgery may be minimally invasive surgery, in which case the surgeon may view the video feed from an endoscope on the display screen 213 showing the surgical site.

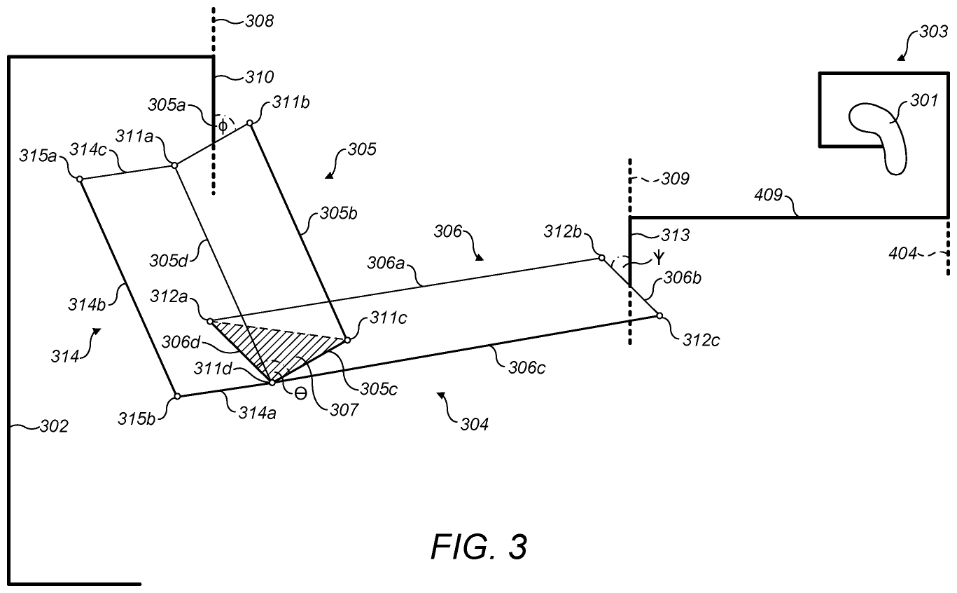

FIG. 3 illustrates an exemplary input device 211 of FIG. 2 in more detail. Input device 211 comprises a hand controller 301 connected to a rigid support structure 302 of the console by a series of articulated links. That series of articulated links comprises a gimbal assembly 303 and an articulated linkage 304. The hand controller 301 is directed connected to the gimbal assembly 303. The gimbal assembly 303 is connected at its distal end to the hand controller 301, and at its proximal end to the articulated linkage 304. The articulated linkage 304 is connected at its distal end to the gimbal assembly 303 and at its proximal end to the support structure 302.

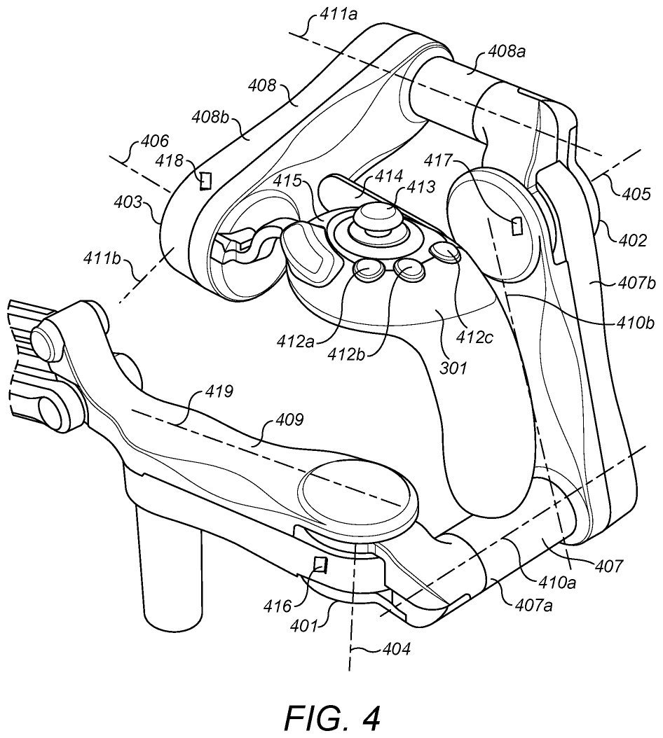

The gimbal assembly is shown in more detail in FIG. 4. The gimbal assembly comprises only three degrees of freedom. These three degrees of freedom are orientations. The three degrees of freedom are provided by three joints: a first joint 401, a second joint 402, and a third joint 403. Each of these three joints is a revolute joint. The first joint 401 connects the terminal link 409 of the articulated linkage 304 to a first link 407 of the gimbal assembly. The first joint 401 permits the first link 407 of the gimbal assembly to rotate relative to the terminal link 409 of the articulated linkage 304 about a first axis 404. The second joint 402 connects the first link 407 of the gimbal assembly to a second link 408 of the gimbal assembly. The second joint 402 permits the second link 408 of the gimbal assembly to rotate relative to the first link 407 of the gimbal assembly about a second axis 405. The second axis 405 is perpendicular to the first axis 404. The third joint 403 connects the second link 408 of the gimbal assembly to the hand controller 301. The third joint 403 permits the hand controller 301 to rotate relative to the second link 408 of the gimbal assembly about a third axis 406. The third axis 406 is perpendicular to the second axis 405.

The first link 407 may be formed of a first portion 407a and a second portion 407b. The first portion 407a is connected to the first joint 401. The second portion 407b is connected to the second joint 402. The first portion 407a and second portion 407b are rigidly connected to each other. The first portion 407a and second portion 407b may not be aligned. For example, as shown in FIG. 4, the longitudinal axis 410a of the first portion 407a may be transverse to the longitudinal axis 410b of the second portion 407b. Axes 410a and 410b may be perpendicular. Thus, the first link 407 as a whole forms an L-shape.

Similarly, the second link 408 may be formed of a first portion 408a and a second portion 408b. The first portion 408a is connected to the second joint 402. The second portion 408b is connected to the third joint 403. The first portion 408a and second portion 408b are rigidly connected to each other. The first portion 408a and second portion 408b may not be aligned. For example, as shown in FIG. 4, the longitudinal axis 411a of the first portion 408a may be transverse to the longitudinal axis 411b of the second portion 408b. Axes 411a and 411b may be perpendicular. Thus, the second link 408 as a whole forms an L-shape.

The articulated linkage 304 and gimbal assembly 303 are arranged such that in every configuration of the articulated linkage and gimbal assembly, the first axis 404 has the same orientation relative to the support structure 302. For example, if the console is located on a horizontal surface, the first axis is vertical in every configuration of the articulated linkage and gimbal assembly. The articulated linkage may be mechanically constrained to cause the first axis 404 to retain the same orientation relative to the support structure. FIG. 3 illustrates a specific example of this.

In FIG. 3, the articulated linkage comprises a parallelogram mechanism. This parallelogram mechanism comprises a first parallelogram 4-bar chain 305 and a second parallelogram 4-bar chain 306. The first parallelogram 4-bar chain 305 comprises links 305a, 305b, 305c and 305d connecting joints 311a, 311b, 311c and 311d. Links 305a and 305c are the same length and maintained parallel. Links 305b and 305d are the same length and maintained parallel. Each of joints 311a, 311b, 311c and 311d is a rotational joint. The axes of rotation of joints 311a, 311b, 311c and 311d are parallel.

The second parallelogram 4-bar chain 306 comprises links 306a, 306b, 306c and 306d connecting joints 312a, 312b, 312c and 311d. Links 306b and 306d are the same length and maintained parallel. Links 306a and 306c are the same length and maintained parallel. Each of the joints 312a, 312b, 312c and 311d is a rotational joint. The axes of rotation of joints 312a, 312b, 312c and 311d are parallel.

Hence, the axes of rotation of all the joints 311a, 311b, 311c, 311d, 312a, 312b and 312c are parallel. Thus, the parallelogram mechanism as a whole is planar.

The whole parallelogram mechanism rotates about axis 308. Axis 308 may be perpendicular to the axes of rotation of the joints. The angle ϕ between link 305a and axis 308 is fixed. The link 305a may rotate about axis 308. Suitably, when the support structure 302 is on a horizontal surface, the axis 308 is vertical. In FIG. 3, link 305a is connected to support structure 302 via link 310. The longitudinal axis of link 310 is axis 308.

The two parallelogram 4-bar chains 305 and 306 are connected by a triangular fixed link 307. That triangular fixed link 307 comprises links 305c and 306d. The angle θ between link 305c and link 306d remains constant. Thus, the orientation of link 306d relative to link 305a is fixed. Thus, the orientation of link 306b relative to link 305a is fixed.

Axis 309 is perpendicular to the axes of rotation of the joints of the parallelogram mechanism. Axis 309 intersects link 306b. The angle Ψ between link 306b and axis 309 is fixed. Thus, axis 308 is maintained parallel to axis 309. In FIG. 3, link 306b is connected to gimbal assembly 303 via a link 313. The longitudinal axis of link 313 is axis 309. In FIG. 3, link 313 is connected to gimbal assembly 303 via terminal link of the articulated linkage 409. Link 409 is connected at one end to link 313, and at the other end to the gimbal assembly 303. In an alternative arrangement, the gimbal assembly 303 may be connected directly to link 313.

The articulated linkage is thereby mechanically constrained to maintain the same orientation between link 305a at one end of the parallelogram mechanism and link 306b at the other end of the parallelogram mechanism. However, the parallelogram mechanism enables movement of link 306b relative to link 305a parallel to the axis 308 and perpendicular to the axis 308, thereby enabling corresponding movement of the hand controller to be accommodated. In the case that the mounting structure 302 is on a horizontal surface, the parallelogram mechanism enables vertical and horizontal motion of the hand controller to be accommodated. Since the parallelogram mechanism can rotate about axis 308 relative to support structure 302, the articulated linkage accommodates all three translational degrees of freedom.

The articulated linkage is constrained to cause axes 308 and 309 to be maintained parallel, whilst enabling the articulated linkage to be moved so as to cause the axes 308 and 309 to move away from each other. In every configuration of the articulated linkage, the first axis 404 has the same orientation relative to the support structure 302. Suitably, the support structure, articulated linkage and gimbal assembly are configured such that when the console is located on a horizontal surface, the first axis 404 is always vertical in every configuration of the articulated linkage and gimbal assembly.

Optionally, the articulated linkage also comprises additional linkage 314. Linkage 314 comprises link 314a, 314b and 314c. Linkage 314 forms a parallelogram with link 305d. Link 314a is connected to link 306c and link 305d by joint 311d. Link 314a is connected to link 314b by joint 315b. Links 314a and 306c may be a single linear bar. In this case, link 306c is fast with respect to link 314a. In other words, link 306c is fixed with respect to link 314a. Link 314b is connected to link 314c by joint 315a. Link 314c is connected to link 305d and link 305a by joint 311a. Suitably, joints 315a and 315b are both rotational joints, having rotational axes which are parallel to the rotation axes of the other joints 311a, 311b, 311c, 311d, 312a, 312b and 312c of the parallelogram mechanism. Links 314a and 314c are the same length and maintained parallel. Links 305d and 314b are the same length and maintained parallel. Thus, links 305b, 305d and 314b are all parallel. Link 314c can rotate with respect to link 305a.

As discussed further below, the articulated linkage 304 may be driven. To achieve this, at least one joint of the first parallelogram 4-bar chain 305 is driven, and at least one joint of the second parallelogram 4-bar chain is driven. Suitably, for first parallelogram 4-bar chain 305, either joint 311a or joint 311b is driven. Driving this single joint causes the whole of parallelogram 4-bar chain 305 to move. An actuator at the driven joint drives the rotation of the joint about its axis. The actuator and joint controller for the driven joint are located near that joint, and hence near axis 308 and the support structure 302.

The second parallelogram 4-bar chain 306 could be driven by actuating any one of joints 312a, 312b, 312c or 311d. These joints are all distal of the support structure 302. An actuator to drive the joint would be located at that joint. This actuator would be reacted by the actuator used to drive the driven joint of the first parallelogram 4-bar chain 305. This would require the actuator of the first parallelogram 4-bar chain 305 to be larger and hence heavier.

The additional linkage 314 enables the second parallelogram 4-bar chain 306 to be driven more efficiently. Specifically, either joint 315a or joint 311a is driven. Driving this single joint causes linkage 314 to move, and hence link 306c to move, and thereby all of second parallelogram 4-bar chain 306 to move. An actuator at the driven joint 315a or 311a drives rotation of that joint about its axis. The actuator and joint controller for the driven joint are located near that joint, and hence near axis 308 and the support structure 302.

Thus, additional linkage 314 enables the articulated linkage 304 to be lighter overall, by enabling more efficient location of the actuators and associated drive electronics to drive the articulated linkage.

Rotation of the hand controller is wholly accommodated by articulation of the joints of the gimbal assembly. A force applied to the hand controller as a roll motion is accommodated by a rotation of the hand controller 301 relative to the second link 408 about the third axis 406. A force applied to the hand controller as a pitch motion is accommodated by a rotation of the second link 408 relative to the first link 407 about the second axis 405. A force applied to the hand controller as a yaw motion is accommodated by a rotation of the first link 407 relative to the terminal link 409 of the articulated linkage about the first axis 404. The first axis 404 being maintained in the same orientation relative to the support structure 302 prevents rotation of the hand controller from being transmitted through to, and hence accommodated by, the articulated linkage 304.

The gimbal assembly may comprise a position sensor 416 located at the first joint 401 for sensing a rotation of the first joint 401 about the first axis 404. The gimbal assembly may comprise a position sensor 417 located at the second joint 402 for sensing a rotation of the second joint 402 about the second axis 405. The gimbal assembly may comprise a position sensor 418 for sensing a rotation of the third joint 403 about the third axis 406. Each position sensor 416, 417, 418 may be configured to transmit its sensed position data to the control unit 214. The control unit 214 may use the received sensed position data to determine the configuration of the gimbal assembly, and thereby the rotational position (i.e. pose/attitude) of the hand controller. Specifically, the control unit 214 may determine: (i) the yaw motion of the hand controller 301 solely from the sensed position data of the position sensor 416 located at the first joint 401, and/or (ii) the pitch motion of the hand controller 301 solely from the sensed position data of the position sensor 417 located at the second joint 402, and/or (iii) the roll motion of the hand controller 301 solely from the sensed position data of the position sensor 418 located at the third joint 403.

The three degrees of freedom of the gimbal assembly are decoupled about the three joints of the gimbal assembly. In other words, at every point in the workspace of the hand controller: (i) the first axis 404 is in the same direction (e.g. vertical) and solely accommodates yaw motion of the hand controller, (ii) the second axis 405 is in the same plane (e.g. horizontal) and solely accommodates pitch motion of the hand controller, and (iii) the third axis 406 is in the same plane (e.g. horizontal) and solely accommodates roll motion of the hand controller. This enables a yaw motion of the hand controller to be measured using only the position sensor 416 on the first joint 401. Similarly, this enables a pitch motion of the hand controller to be measured using only the position sensor 417 on the second joint 402. Similarly, this enables a roll motion of the hand controller to be measured using only the position sensor 418 on the third joint 403. For a four degree of freedom gimbal assembly, detecting one of yaw, pitch and roll motion of the hand controller requires compound measurements from a plurality of sensors. Thus, the gimbal assembly described herein enables a more computationally efficient calculation to be performed by the control unit to determine the configuration of the gimbal assembly.

Translation of the hand controller is accommodated by articulation of the joints of the articulated linkage 304. A force applied to the hand controller so as to translate the hand controller directly towards the support structure 302 or parallel to the axis 308 is accommodated by rotation of the joints of the parallelogram mechanism about their axes. A force applied to the hand controller so as to translate the hand controller in a direction transverse to the direction of the support structure 302 is accommodated by rotation of the articulated linkage about the axis 308. It is also accommodated by a small rotation of the gimbal assembly about the first axis 404 in order to maintain the alignment of the gimbal assembly.

The articulated linkage 304 may comprise a position sensor 314 located at each joint for sensing rotation of that joint about its axis. Each position sensor 314 may be configured to transmit its sensed position data to the control unit 214. The control unit 214 may use the received sensed position data to determine the configuration of the articulated linkage, and thereby the translational position of the hand controller. Specifically, the control unit 214 may use the sensed position data received from sensors 314, and the dimensions of the articulated linkage 304 and gimbal assembly 303 to determine the location of the hand controller 301 in the workspace in which the hand controller 301 is permitted to move.

Any compound motion resulting from forces applied to the hand controller can be resolved into the six force components described above: roll, pitch and yaw motions of the hand controller, and translation in three perpendicular directions. Each of those force components is accommodated, and sensed, as described above.

By decoupling the joints that accommodate rotational motion of the hand controller (i.e. the gimbal assembly) from the joints that accommodate translational motion of the hand controller (i.e. the articulated linkage), the correspondence experienced by the user between the direction of rotation and motion of the hand controller and that of the end effector (as displayed on the console display) is independent of the position of the hand controller within the workspace of the hand controller.

The articulated linkage arrangement shown in FIG. 3 is an example. The articulated linkage may comprise alternative or further links and joints, and still be mechanically constrained so as to cause the first axis 404 to retain its orientation relative to the support structure. For example, instead of the parallelogram mechanism described above, the articulated linkage may comprise a scissor arm mechanism mounted on a rotation axis, a sarrus linkage mechanism mounted on a rotation axis, or a combination of a scissor arm mechanism and a sarrus linkage mechanism.

The hand controller 301 comprises several inputs. For example, FIG. 4 illustrates push-buttons 412a, 412b, 412c and joystick 413. The hand controller 301 may also include an input lever or trigger 414. The user can depress the input lever 414 towards the body 415 of the hand controller 301. Further exemplary inputs include rotational knobs and rocker switches.

As mentioned above, the control unit 214 controls the robot arm 203 in response to control inputs from input device 211, and optionally additionally from other sources such as position sensors and/or force/torque sensors on the robot arm. The control inputs from input device 211 may include: (i) control inputs from the inputs on the hand controller, for example button pushes, input lever movement, and/or (ii) control inputs from the gimbal assembly resulting from rotation of the hand controller, and/or (iii) control inputs from the articulated linkage resulting from translation of the hand controller.

The code executed by the processor 215 of control unit 214 is configured so that the motion of the robot is primarily dictated by the inputs from the input device 211. For example, in normal operating mode: (i) the attitude of the end effector 207 may be set by the attitude of the hand controller about its rotational degrees of freedom as determined from the control inputs from the gimbal assembly; (ii) the position of the end effector 207 may be set by the position of the hand controller about its translational degrees of freedom as determined from the control inputs from the articulated linkage; and (iii) the configuration of the jaws of the end effector 207 may be set by the position of the input lever 414 relative to the body 415 of the hand controller.

The gimbal assembly illustrated in FIG. 4 only has three degrees of freedom to govern motion in three dimensions. This enables the gimbal assembly to be smaller and lighter than those which have a redundant degree of freedom, i.e. four degrees of freedom in total. However, a redundant degree of freedom is useful in avoiding the gimbal assembly from reaching a kinematic singularity. A kinematic singularity occurs when the gimbal assembly adopts a configuration that prevents it from being able to rotate in a particular direction. For a gimbal assembly with only three degrees of freedom this can happen when two axes of the gimbal assembly align. For example, in FIG. 4, if the second link 408 is rotated about the second axis 405 by 90°, then the first axis 404 becomes aligned with the third axis 405. In this configuration, the hand controller can only be rotated about two axes, not three. A four degree of freedom gimbal assembly avoids this problem by providing a redundant degree of freedom. Thus, even if two axes become aligned, the hand controller is still able to be rotated about three axes.

The range of motion of each of the joints of the gimbal assembly may be limited so as to prevent the gimbal assembly from adopting a configuration which results in a kinematic singularity. The limits of the range of motion of each joint of the gimbal assembly will now be described with reference to a central position of the gimbal assembly. FIG. 4 illustrates a gimbal assembly in the central position. In this central position, the first axis 404, second axis 405 and third axis 406 are all perpendicular to each other. In the central position, a longitudinal axis 419 of the terminal link 409 of the articulated linkage may be parallel with the third axis 406. In the central position, the third joint 403 may be at the midpoint in its range of motion.

From the central position, the range of motion of the first joint 401 may be limited such that it is capable of rotating more than 90° in either rotational direction about the first axis 404. From the central position, the maximum rotation angle of the first joint 401 may be between 90° and 125° in a rotational direction which causes the first link 407 to move towards the distal end 409 of the articulated linkage. Preferably, the maximum rotation angle of the first joint is between 90° and 115° in this rotational direction. The maximum rotation angle of the first joint 401 may be 115° in this rotational direction. From the central position, the maximum rotation angle of the first joint 401 may be between 90° and 110° in a rotational direction which causes the first link 407 to move away from the distal end 409 of the articulated linkage. Preferably, the maximum rotation angle of the first joint is between 90° and 100° in this rotational direction. The maximum rotation angle of the first joint 401 may be 100° in this rotational direction.

Suitably, the range of motion of the first joint 401 about the first axis 404 in either rotational direction is increased beyond 90° in order to accommodate the change in orientation of the articulated linkage 304 when the hand controller undergoes a translation motion. By doing so, the angular range of motion of the gimbal assembly is not affected by the location of the gimbal assembly in the workspace of the hand controller.

From the central position, the range of motion of the second joint 402 may be limited such that it is capable of rotating less than 90° in either rotational direction about the second axis 405. From the central position, the maximum rotation angle of the second joint 402 may be between 70° and 90° in a rotational direction which causes the second link 408 to move towards the first link 407. Preferably, the maximum rotation angle of the second joint is between 80° and 90° in this rotational direction. The maximum rotation angle of the second joint 402 may be 80° in this rotational direction. From the central position, the maximum rotation angle of the second joint 402 may be between 70° and 90° in a rotational direction which causes the second link 408 to move away from the first link 407. Preferably, the maximum rotation angle of the second joint is between 80° and 90° in this rotational direction. The maximum rotation angle of the second joint 402 may be 80° in this rotational direction.

Suitably, the range of motion of the second joint 402 about the second axis 405 in either rotational direction is limited below 90° in order to prevent the first axis 404 and third axis 406 from aligning (which would happen at a rotation angle of 90° about the second axis 405) and thereby causing a kinematic singularity.

From the central position, the range of motion of the third joint 403 may be limited such that it is capable of rotating less than or the same as 90° in either rotational direction about the third axis 406. From the central position, the maximum rotation angle of the third joint 403 may be between 80° and 90° in a rotational direction which causes the hand controller 301 to move towards the second link 408. Preferably, the maximum rotation angle of the third joint is 90° in this rotational direction. From the central position, the maximum rotation angle of the third joint 403 may be between 80° and 90° in a rotational direction which causes the hand controller 301 to move away from the second link 408. Preferably, the maximum rotation angle of the third joint is 90° in this rotational direction.

Although the joint limits described above limit the range of motion of the joints, that motion is still sufficient to accommodate the full range of motion of the human wrist. Because the hand controller 301 is being manipulated by a human hand, the user does not experience a limit to the available range of motion, since they reach the limit of the range of motion of their hand before reaching the limit of a range of motion of a joint of the gimbal assembly.

In addition to the range of motion limits described above, constraining the first axis 404 to be in the same orientation relative to the support structure 302 (e.g. vertical), ensures that the user is able to rotate the hand controller in both directions about each of the first, second and third axes. If the first axis 404 was not constrained in this way, then in some configurations of the articulated linkage 304, from its central position the gimbal assembly would be closer to a joint limit in one rotational direction about an axis than the opposing rotational direction, thereby causing the range of motion to be more limited in one rotational direction than the opposing rotational direction about the axis.

FIG. 4 illustrates a hand controller for manipulation by the right hand of a user. The console may instead, or additionally, comprise a hand controller (and associated gimbal assembly and articulated linkage) for manipulation by the left hand of a user. The hand controller, gimbal assembly and articulated linkage for the left hand of a user would be a mirror image of the arrangement described above with respect to the right hand of a user. In the case that the console comprises two hand controllers (and associated gimbal assemblies and articulated linkages), one hand controller for manipulation by the right hand of a user may, via control unit 214, control manipulation of a first robot arm and instrument, and the other hand controller for manipulation by the left hand of a user may, via control unit 214, control manipulation of a second robot arm and instrument.

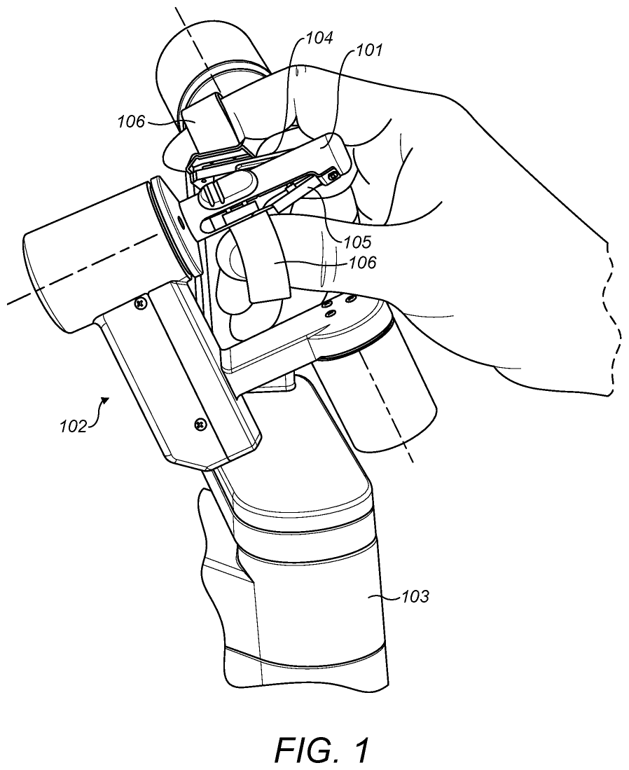

The gimbal assembly described herein is smaller and lighter than the four degree of freedom gimbal assembly shown in FIG. 1. This enables easier usability and greater flexibility in operation, particularly when two hand controllers are being manipulated in the same workspace by the user. For example, a user manipulating two hand controllers as described herein in the same workspace may be able to cross their hands over in the workspace (due to the compact nature of the associated gimbal assemblies and articulated linkages) which is not possible with the arrangement shown in FIG. 1.

In the apparatus described herein, the gimbal assembly 303 and articulated linkage 304 are articulated directly by force applied to the hand controller 301 by a user. The joints of the articulated linkage 304 and/or the joints of the gimbal assembly 303 may additionally be driven. The joints may be driven in order to: (i) compensate for gravity acting on the joints, and/or (ii) cause the joints to maintain a pose so as to feel weightless to the user. The joints may also be driven so as to provide haptic feedback to the user. This haptic feedback may be, for example force feedback via the hand controller pushing the user's hand. The haptic feedback may be a vibration, rumble or click transmitted to the user's hand via the hand controller. The joints are not otherwise driven. The first axis 404 is maintained in the same orientation relative to the support structure 302 of the console by mechanically constraining the articulated linkage 304. In an alternative implementation, the joints of the articulated linkage 304 could instead be driven in response to sensed forces applied to the hand controller 301. In this alternative implementation, the joints of the articulated linkage 304 could be driven in such a way that the first axis 404 is always maintained in the same orientation relative to the support structure 302.

The robot described herein may be a surgical robot having a surgical instrument attachment with a surgical end effector. Alternatively, the robot could be an industrial robot or a robot for another function. The instrument could be an industrial tool.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A console configured to control a robotic manipulator having an end effector, the console comprising:
a hand controller connected to a gimbal assembly; and
an articulated linkage connected at its proximal end to a rigid support structure, and at its distal end to the gimbal assembly;
wherein the gimbal assembly comprises only three degrees of freedom provided by only three joints, a first joint of the three joints permitting the gimbal assembly to rotate relative to the distal end of the articulated linkage about a first axis; and
wherein the articulated linkage and gimbal assembly are arranged such that in every configuration of the articulated linkage and gimbal assembly, the first axis has the same orientation relative to the support structure, wherein the articulated linkage has a parallelogram profile thereby mechanically constraining the first axis to have the same orientation relative to the support structure in every configuration of the articulated linkage.

2. A console as claimed in claim 1, configured such that when the console is located on a horizontal surface, the first axis is vertical in every configuration of the articulated linkage and gimbal assembly.

3. A console as claimed in claim 1, configured to wholly accommodate rotation of the hand controller by articulation of the three joints of the gimbal assembly.

4. A console as claimed in claim 1, configured to accommodate translation of the hand controller by articulation of the articulated linkage.

5. A console as claimed in claim 1, wherein the gimbal assembly comprises:
a first link and a second link;
a second joint permitting the first link to rotate relative to the second link about a second axis, the second axis being perpendicular to the first axis; and
a third joint permitting the hand controller to rotate relative to the second link about a third axis, the third axis being perpendicular to the second axis.

6. A console as claimed in claim 5, wherein from a central position of the gimbal assembly in which the first axis, second axis and third axis are all perpendicular to each other, the range of motion of the first joint is limited such that it is capable of rotating more than 90° in either rotational direction about the first axis.

7. A console as claimed in claim 6, wherein from the central position of the gimbal assembly, the first joint is limited to a maximum rotation angle of between 90° and 115° in a rotational direction which causes the first link to move towards the distal end of the articulated linkage.

8. A console as claimed in claim 6, wherein from the central position of the gimbal assembly, the first joint is limited to a maximum rotation angle of between 90° and 100° in a rotational direction which causes the first link to move away from the distal end of the articulated linkage.

9. A console as claimed in claim 5, wherein from a central position of the gimbal assembly in which the first axis, second axis and third axis are all perpendicular to each other, the range of motion of the second joint is limited such that it is capable of rotating less than 90° in either rotational direction about the second axis.

10. A console as claimed in claim 9, wherein from the central position of the gimbal assembly, the second joint is limited to a maximum rotation angle of between 80° and 90° in a rotational direction which causes the second link to move towards the first link.

11. A console as claimed in claim 8, wherein from the central position of the gimbal assembly, the second joint is limited to a maximum rotation angle of between 80° and 90° in a rotational direction which causes the second link to move away from the first link.

12. A console as claimed in claim 5, wherein from a central position of the gimbal assembly in which the first axis, second axis and third axis are all perpendicular to each other, the range of motion of the third joint is limited such that it is capable of rotating less than or the same as 90° in either rotational direction about the third axis.

13. A console as claimed in claim 12, wherein from the central position of the gimbal assembly, the third joint is limited to a maximum rotation angle of 90° in either rotational direction about the third axis.

14. A console as claimed in claim 1, further comprising a position sensor located at the first joint configured to measure a yaw motion of the hand controller solely by sensing a rotation of the first joint about the first axis.

15. A console as claimed in claim 5, further comprising a position sensor located at the second joint configured to measure a pitch motion of the hand controller solely by sensing a rotation of the second joint about the second axis.

16. A console as claimed in claim 5, further comprising a position sensor located at the third joint configured to measure a roll motion of the hand controller solely by sensing a rotation of the third joint about the third axis.

17. A console as claimed in claim 1, the console being a surgeon's console configured to control a surgical robot carrying a surgical instrument.

18. A console as claimed in claim 1, configured to control a further robotic manipulator having a further end effector, the console further comprising:
a further hand controller connected to a further gimbal assembly; and
a further articulated linkage connected at its proximal end to the rigid support structure, and at its distal end to the further gimbal assembly;
wherein the further gimbal assembly comprises only three degrees of freedom provided by only three joints, a first joint of the three joints permitting the further gimbal assembly to rotate relative to the distal end of the further articulated linkage about a fourth axis; and
wherein the further articulated linkage and the further gimbal assembly are arranged such that in every configuration of the further articulated linkage and the further gimbal assembly, the fourth axis has the same orientation relative to the support structure.

19. A console as claimed in claim 18, wherein the hand controller is configured for operation by one hand of a user, and the further hand controller is configured for operation by the other hand of the user.
